(19) United States
(12) Patent Application Publication
(10) Pub. No.: US 2015/0209546 A1
(43) Pub. Date: Jul. 30, 2015

(54) Anesthesia Machine

(71) Applicants: Douglas Steven PERNIKOFF, (US); Terry Michael POST, Sullivan, MO (US); PORTABLE ANESTHESIA SOLUTIONS LLC, Chesterfield, MO (US)

(72) Inventors: Douglas Steven Pernikoff, Saint Louis, MO (US); Terry Michael Post, Chesterfield, MO (US)

(21) Appl. No.: 14/427,277

(22) PCT Filed: Sep. 10, 2013
(86) PCT No.: PCT/US2013/059100

§ 371 Date: Mar. 10, 2015

Related U.S. Application Data
(60) Provisional application No. 61/743,711, filed on Sep. 10, 2012.

(51) Int. Cl. A61M 16/12; A61M 16/18; A61M 16/08; A61M 16/00

(52) U.S. Cl. CPC A61M 16/125; A61M 16/0003; A61M 16/18; A61M 16/0816; A61M 16/0051; A61M 2016/0033; A61M 2205/3375; A61M 2205/3368; A61M 2205/3653; A61M 2205/0272; A61M 2205/505; A61M 2205/52; A61M 2230/205; A61M 2230/04; A61M 2205/3584; A61M 2205/3303; A61M 2202/0208; A61M 2202/0283; A61M 2202/025; A61M 2202/048; A61M 2230/432; A61M 2230/437

(57) ABSTRACT

Disclosed is an anesthesia gas delivery device and methods of use therefor. The device comprises an aluminum cover plate, a gas inlet for a carrier gas, a gas outlet for a diluent anesthetic gas, a gas corridor in fluid communication with and extending between the gas inlet and the gas outlet, at least four ultrasound acoustic sensors, at least 2 thermistors, a reservoir comprising a reservoir housing for a liquid inhalational anesthetic, a controller, a graphical user interface, and means for transferring a sample of a liquid inhalational anesthetic from the reservoir to the gas corridor. Methods of providing anesthesia to a subject using the device are also disclosed. These methods comprise using ultrasound and temperature data to determine anesthetic gas velocity, composition and concentration.

### CROSS REFERENCE TO RELATED APPLICATION

This application claims priority to U.S. Provisional Application Ser. No. 61/743,711 filed Sep. 10, 2012, which is incorporated herein by reference in its entirety.

### FIELD

This work relates generally to devices for controlling anesthesia.

### INTRODUCTION

An anesthetic, or combination of anesthetics, may be delivered to a patient in order to produce the effects of sedation, analgesia, and neuro-muscular block, broadly referred to as anesthesia. Ditterent anesthetics produce different effects and degrees of effects, and therefore, must be carefully delivered to the patient. Under established methods, a carrier gas (or a combination of carrier gases) is passed over a liquid inhalation anesthetic (or a combination of anesthetics) in a vaporizer, for delivery to the patient.

Determining the composition of an anesthetic gas mixture is critical for successful anesthesia. Ultrasound sensors have been used for this purpose in conjunction with a vaporizer (e.g., US Patent Application Publication 2012/0240928 of Bottom). However, anesthesia machines with greater portability are needed.

### SUMMARY

The present inventors have developed an anesthesia machine. In various embodiments, the device can be portable and can be used to support pain management in various environments, such as, without limitation, emergency transport vehicles, outpatient facilities, and hospitals including military field hospitals. In various embodiments, an anesthesia machine of the present teachings, which utilizes inhalational anesthetics, can be used as an alternative to the administration of opiates or other pharmaceuticals for management of pain.

In various embodiments, an anesthesia machine of the present teachings has graduated output that can be substantially more accurate and reliable under changing environmental applications compared to other commonly used anesthesia machines. In various configurations, a device of the present teachings comprises acoustic ultrasound sensors, which can be used to measure gas velocity and determine gas composition (including species of gases and concentration). An ultrasound sensor of the present teachings can function as a microphone, a speaker, or a combination thereof. In some embodiments, acoustic sensors can be situated at known distances from each other for time-of-flight determinations of ultrasound signals. In various configurations, time-of-flight measurements can be used along with temperature measurements by thermistors, thereby allowing determination and monitoring of composition, concentration and flow rate of an anesthetic gas mixture such as, for example and without limitation, a carrier gas such as oxygen, nitrous oxide, air, helium or a combination thereof, mixed with an inhalational anesthetic such as, for example and without limitation, sevoflurane, desflurane, isoflurane, halothane, methoxyflurane, ethrane or ether.

In some embodiments, an anesthesia machine can be used to determine and monitor composition, concentration and flow rate of exhalation gases, e.g., during a surgical procedure. Medical personnel such as, for example, an anesthesiologist can use the device to monitor and adjust depth of anesthesia.

In some embodiments, the present teachings include an anesthesia gas delivery device that comprises a cover plate, a gas inlet for a carrier gas, a gas outlet for a diluent anesthetic gas, a gas corridor in fluid communication with and extending between the gas inlet and the gas outlet, a first acoustic sensor situated in the gas corridor adjacent to the gas inlet, a second acoustic sensor situated in the gas corridor downstream of the first acoustic sensor, a third acoustic sensor situated in the gas corridor downstream from the second acoustic sensor, and a fourth acoustic sensor situated in the gas corridor downstream from the third acoustic sensor and adjacent to the gas outlet. In various configurations, the corridor can be “U” shaped. In some embodiments, a corridor can comprise an array of parallel micro tubes. In some configurations, these tubes can be positioned between the first and second acoustic sensors, and can be used to induce laminar flow in gas passing through the corridor. In various embodiments, a device of the present teachings includes a reservoir comprising a housing for a liquid inhalational anesthetic. In various configurations, a reservoir can comprise an inhalational anesthetic such as, without limitation, sevoflurane, desflurane, isoflurane, halothane, methoxyflurane, ethrane or ether, and can have a capacity of from about 5 ml to about 30 ml. In various configurations, a wall of the reservoir can include one or more grooves that can conduct migration of a liquid inhalational anesthetic towards a liquid transfer means for introducing an inhalational anesthetic to the gas corridor, as discussed below. In various configurations, grooves can be etched grooves. In some configurations, a reservoir can comprise a resistive wire, which can be used to determine volume of liquid anesthetic in the reservoir. In some configurations, either or both of the gas inlet and the gas outlet can each comprise a barbed hose connector. In some configurations, a barbed hose connector can be a retractable barbed hose connector.

Embodiments of the present teachings include means for transferring a sample of a liquid inhalational anesthetic from a reservoir to a gas corridor. In some configurations, such means can be positioned between the second acoustic sensor and the third acoustic sensor. Such means can include a ferromagnetic (e.g., ferrite, stainless steel or chromed iron) rod or bar having a slot or trough. In various configurations, the rod or bar can be cylindrical or rectangular in shape. A solenoid can be used to move the slotted rod or bar to a position where the slot is in liquid communication with a portal that allows a liquid from the reservoir to fill the slot. In various configurations, the rod or bar can be supported by springs such as steel springs. The solenoid, which can be controlled by the controller, can be used to move the rod or bar to a position where the slot is in liquid communication with a portal that allows a liquid from the slot to mix with carrier gas in the corridor. In various embodiments, liquid is unable to flow from the slot to the corridor while the slot is positioned to fill with liquid from the reservoir; liquid is unable to flow from the reservoir to the slot while the slot is positioned to release liquid to the corridor. In various configurations, one cycle of movement of the rod or bar transfers one slot volume of liquid from the reservoir to the corridor. In various configurations, the volume of liquid transferred in one cycle can be from 1 to 30 microliters, for example, about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters, about 25 microliters, about 26 microliters, about 27 microliters, about 28 microliters, about 29 microliters or about 30 microliters. In some configurations, the volume of liquid transferred in one cycle can be 1.74 microliters. In some configurations, repeated electrical pulses to the solenoid can be used to introduce multiples of unit volumes of liquid inhalational anesthetic, wherein the unit volume is determined by the size of the slot.

Upon introduction of liquid anesthetic to the corridor, the anesthetic can be vaporized by vaporizing means, such as contact with flowing carrier gas. In some configurations, means for vaporizing an anesthetic can include a providing a heat source such as a heat patch or resistive wire in addition to or instead of carrier gas flow.

In various embodiments, an anesthesia machine of the present teachings can include an electronic controller, which can include internet communications hardware and software which allow control from a remote location. A controller can receive data from the acoustic sensors and thermistors. In various configurations, a controller can not only determine the composition, concentration and velocity of carrier gas and diluent gas based on the input data, it can also allow medical personnel (such as an anesthesiologist or emergency medical technician) to adjust gas flow rates, and also adjust amount of liquid inhalational anesthetic added to the corridor, and thereby modify diluent anesthetic gas composition and/or concentration. In some configurations, a controller can include alarm limits which can, for example, automatically reduce amount of anesthetic in the diluent gas, and/or automatically alert medical personnel of a change in respiration or reduction in amount of liquid inhalational anesthetic in a reservoir below a predetermined alarm limit.

In various configurations, the first acoustic sensor can function as a microphone and can report velocity of a carrier gas at the gas inlet. In various embodiments, time-of-flight measurements between the first and the second acoustic sensors can be used to determine composition, concentration and velocity of gas upstream from the means for introducing an inhalational anesthetic. In some embodiments, a first thermistor positioned between the first and second acoustic sensors can also be used to determine composition, concentration and velocity of gas upstream from the means for introducing an inhalational anesthetic. In various configurations, the third sensor produces a third sensor signal indicative of composition of gas downstream from the means for introducing an inhalational anesthetic, the fourth acoustic sensor produces a fourth signal indicative of composition of diluent gas at the gas outlet. In various embodiments, time-of-flight measurements between the third and the fourth acoustic sensors can be used to determine composition, concentration and velocity of gas downstream from the means for introducing an inhalational anesthetic. In some embodiments, a second thermistor positioned between the third and fourth acoustic sensors can also be used to determine composition, concentration and velocity of gas downstream from the means for introducing an inhalational anesthetic. In various configurations, the controller receives the first, second, third and fourth sensor signals, as well as thermal data from the first and second thermistors, and computes a composition and concentration of diluent anesthetic gas. In some configurations, differences in temperatures measured by the thermistors can be used to aid determination of diluent gas composition and concentration. In some configurations, a controller can be configured to receive data from resistive wire that indicate volume of liquid inhalational anesthetic remaining in a reservoir.

In various embodiments, a device of the present teachings can be housed in aluminum and/or a hard plastic such as a co-polymer resin. In various configurations, the corridor can be substantially square, rectangular, circular or elliptical in cross section, and can be, for example, substantially rectangular, e.g., 7 mm across×4 mm deep.

In various embodiments, a device of the present teachings can include a graphical user interface (GUI), such as a capacitive touch screen. In various configurations, the GUI display can comprise one or more of carrier gas composition, inhalational anesthetic species and percentage in diluent gas, flow rate, exhalation gas composition, exhalation gas concentration, exhalation gas flow rate, “3 lead” electrocardiology data and SpO₂ data. In some configurations, a GUI can be a 180 mm×130 mm capacitive touch screen.

In various embodiments, a device of the present teachings can include a USB port such as a micro USB port.

In various embodiments, a device of the present teachings can include connectors for SpO₂ leads.

In various embodiments, a device of the present teachings can include connectors for electrocardiography leads.

In various embodiments, a device of the present teachings can include a battery to power the device.

In various embodiments, a device of the present teachings can include a second corridor configured to receive exhaled gas, a fifth acoustic sensor, a sixth acoustic sensor and a third thermistor. In various configurations, these sensors and thermistor can be used to determine composition of exhaled gas. In various configurations, medical personnel such as an anesthesiologist can determine depth of anesthesia and adjust anesthetic amounts based on exhaled gas composition.

The present teachings include a device for transferring a pre-determined volume of liquid from a reservoir to a receiving chamber. A device of these embodiments can include a ferromagnetic (e.g., ferrite, stainless steel or chromed iron) rod or bar having a slot or trough. In various configurations, the rod or bar can be cylindrical or rectangular in shape. A solenoid can be used to move the slotted rod or bar to a position where the slot is in liquid communication with a portal that allows a liquid from the reservoir to fill the slot. In various configurations, the rod or bar can be supported by springs such as steel springs. The solenoid, which can be controlled by a controller, can be used to move the rod or bar to a position where the slot is in liquid communication with a portal that allows a liquid from the slot to flow into the receiving chamber. In various embodiments, liquid is unable to flow from the slot to the receiving chamber while the slot is positioned to fill with liquid from the reservoir; liquid is unable to flow from the reservoir to the slot while the slot is positioned to release liquid to the receiving chamber. In various configurations, one cycle of movement of the rod or bar transfers one slot volume of liquid from the reservoir to the receiving chamber. In various configurations, the volume of liquid transferred in one cycle can be from 1 to 30 microliters, for example, about 1 microliter, about 2 microliters, about 3 microliters, about 4 microliters, about 5 microliters, about 6 microliters, about 7 microliters, about 8 microliters, about 9 microliters, about 10 microliters, about 11 microliters, about 12 microliters, about 13 microliters, about 14 microliters, about 15 microliters, about 16 microliters, about 17 microliters, about 18 microliters, about 19 microliters, about 20 microliters, about 21 microliters, about 22 microliters, about 23 microliters, about 24 microliters, about 25 microliters, about 26 microliters, about 27 microliters, about 28 microliters, about 29 microliters or about 30 microliters. In some configurations, the volume of liquid transferred in one cycle can be 1.74 microliters. In some configurations, repeated electrical pulses to the solenoid can be used to introduce multiples of unit volumes of a liquid such as, e.g., a liquid inhalational anesthetic, wherein the unit volume is determined by the size of the slot.

Embodiments of the present teachings include methods of performing anesthesia on a subject. In various configurations, these methods include mixing a carrier gas with an inhalational anesthetic using a device described herein to form a diluent gas; and supplying the diluent gas to the subject. The diluent gas can be supplied to the subject by methods and using materials well known to skilled artisans.

In various configurations, the carrier gas can be, without limitation, oxygen, nitrous oxide, air, helium or a combination thereof. In various configurations, the inhalational anesthetic can be, without limitation, sevoflurane, desflurane, isoflurane, halothane, methoxyflurane, ethrane or ether.

In various configurations, the methods can also include evaluation of exhalation gas, which can include, e.g., composition of the exhalation gas and flow rate of exhalation gas. Using a device described herein, medical personnel such as an anesthesiologist can view “real-time” data about the anesthesia including anesthetic composition and flow rate, as well as “real-time” patient data such as electrocardiography, pulse rate, breathing rate, CO₂ output, and the like.

### BRIEF DESCRIPTION OF THE DRAWINGS

FIG. 1 illustrates an embodiment of an anesthesia machine described here.

FIG. 2 illustrates the device without the cover.

FIG. 3 illustrates a diagrammatic view of the device, highlighting an array of parallel micro tubes.

FIG. 4 illustrates a means for transferring a pre-determined volume of liquid from a reservoir to a receiving chamber such as a corridor of the present teachings.

FIG. 5 illustrates a portion of a device of the present teachings, including a “feedback” channel for analyzing exhalation gases.

### DETAILED DESCRIPTION

The present inventors have developed an anesthesia machine that, in various embodiments, uses a novel means for introducing a liquid inhalational anesthetic to a carrier gas to form a diluent gas. The device in various configurations can be used to introduce a liquid inhalational anesthetic to a carrier gas in quantized amounts. A controller comprising a graphical user interface (GUI) capacitive touch screen can display “real time” physiological data and provide user controls of anesthesia.

In various embodiments, an anesthesia machine of the present teachings can be a portable anesthesia gas delivery device that has a graduated output that can be substantially more accurate and reliable under changing environmental conditions compared to existing anesthesia machines. By using ultrasound acoustic sensors spaced at known distances from each other and in contact with a gas moving through a corridor, time-of-flight data can be combined with temperature measurements using thermistors to determine the composition, velocity and temperature of carrier gas and diluent gas. The data can be used to compute and adjust the frequency of a flat solenoid that controls transport a micro drop of liquid inhalational anesthetic into the gas corridor where it can evaporate and join the flow of carrier gas. The sensors can also detect the combined composition prior to exit of the machine based in part by the SOS (speed of Sound), temperature and the changes therein.

In some configurations, an anesthesia machine of the present teachings can have dimensions of approximately 1 inch thickness, approximately 7 inches in length, and approximately 5 inches in width. In some configurations, distance between acoustic sensors for time-of-flight measurements can be, for example and without limitation, about 100 mm, or 100.63 mm, or 99.99 mm.

In some configurations, means for transferring a sample of a liquid such as an inhalational anesthetic from a reservoir to a receiving chamber such as a gas corridor include the use of a ferritic bar or cylinder comprising a slot or trough. In some configurations, the position of the bar or cylinder can be controlled by a solenoid such as a “flat” solenoid.

In some configurations, an anesthesia machine of the present teachings can comprise a digital controller, which can be a microcontroller with sufficient clock speed to accurately evaluate the transducted waves of sound through a corridor (such as a corridor of aluminum). In some configurations, the control can allow for a large ratio of delta measurements between events.

In some configurations, an anesthesia machine of the present teachings can comprise acoustic sensors. Such sensors can transmit and/or receive ultrasound, and can comprise graphene. In some configurations, a sensor can have low impedance, and can be formed on a 3-D printer. In some configurations, an anesthesia machine of the present teachings can comprise ultrathin inductor coils of printed lamina which are capable of inducing an electric field powerful enough to affect a miniature disk of coated steel. In some configurations the induction coils can be fixedly attached to a thin sheet of polyvinyl chloride located at the center of the laminated coil whose bottom can be exposed to the flowing gases.

In some configurations, an anesthesia machine of the present teachings can detect the presence, velocity and temperature of user supplied gases introduced into the device by deductive algorithms based on 6 sensor points throughout the flow corridor. In various configurations, data obtained from the sensor points can be compared to known “signatures” whereby identity of the carrier gas as well as the percent by volume of the combined gases can be determined.

In some configurations, an anesthesia machine of the present teachings can comprise an oscillator of sufficient speed such that by counting the number of clock cycles between transmit and receive, an acoustic signal can be detected and the gases can be determined with a large margin per percent available as a function of the computers speed.

In some configurations, an anesthesia machine of the present teachings can comprise a flow corridor that can take in a carrier gas to which can be added liquid inhalational anesthetic in quantized volumes of about 1 microliter up to about 30 microliters. In some configurations, liquid inhalational anesthetic can be introduced at a central point of the corridor, thereby allowing the downstream portion of the corridor to give rise to combinant gases before exit.

In some configurations, an anesthesia machine of the present teachings can comprise longitudinal microgauge aluminum tubes situated in the inlet portion of the flow corridor. In various aspects, the presence of the microgauge tubes can force a laminar flow of incoming carrier gas.

In some configurations, an anesthesia machine of the present teachings can comprise at least 4 fixedly attached acoustic sensors. In various configurations, these sensors can be capable of transmitting a signal or receiving a signal; the function of a sensor can be defined by the pin data of the controller.

In some configurations, in an anesthesia machine of the present teachings, acoustic signals of an incoming carrier gas can be analyzed to deduce how close the gas is to a reference gas such as pure oxygen.

In some configurations, an anesthesia machine of the present teachings can be capable of accepting an input from the user and computing the cycle frequency of the delivery solenoid which can mechanically reach up and grab a microliter drop of the liquid inhalational anesthetic and deliver it to the flow corridor where it can evaporate and join the carrier stream towards the exit.

In some embodiments, an anesthesia machine of the present teachings can be capable of maintaining a sufficient supply of heat for the highest user demand rate of evaporation by “dry firing” the delivery solenoid such that no liquid is transmitted but friction can induce heat to the surrounding body of aluminum.

In some embodiments, an anesthesia machine of the present teachings can comprise a substantially flat bar of ferrous material with a single micro slot or trough that is positioned such that when exposed to an attracting electric field, momentarily over opposes two flat serpentine pieces of high memory wire, allowing the slot or trough to soundlessly travel between the closed position and open conducting a drop of liquid from one chamber to another.

In some embodiments, an anesthesia machine of the present teachings can comprise a graphical user interface on the front while the back surface of the same sheets of glass can enclose the liquid and flow chamber.

In some configurations, an anesthesia machine of the present teachings can be capable of being fully controlled from anywhere on earth by a user such as a licensed medical practitioner via high band width internet embedded in the computer of the device.

In some configurations, an anesthesia machine of the present teachings can comprise means of gathering patient physiological data pertinent to safe surgical anesthesia such as electrocardiography ECG, pulse, respiration, EtC02 and temperature. The means can include storing the data on the controller.

In some configurations, an anesthesia machine of the present teachings can record relative barometric pressure during the start up phase of carrier gas velocity and the signal conduction time as a function of temperature; measured by both thermisters and by comparison to the ideal gas equations. In some configurations, elevation can be incorporated for further accuracy by a GPS rf receiver.

In some configurations, an anesthesia machine of the present teachings can acquire, report, and/or record patient thoracic impedance as it changes through a surgical procedure. Furthermore, in some configurations, an anesthesia machine of the present teachings can provide an alarm condition for the operator which can thereby add another layer of observational vigilance during a case.

In some embodiments, an anesthesia machine of the present teachings can comprise a single resistive wire within the liquid reservoir whose impedance changes as the liquid level drops, and can thereby provide real time digital output for the user.

In some configurations, an anesthesia machine of the present teachings can comprise a luer lock system for adding liquid agent such that it can allow in flowing room air to prevent a negative pressure head on the liquid but can have a one-way liquid escape flap to prevent a liquid inhalational anesthetic from leaking during unit inversion.

In some embodiments, an anesthesia machine of the present teachings can comprise a second corridor through which expired gases are able to flow through with minimal resistance. In some configurations, this secondary corridor can contain a spaced pair of laminated inductor coils separated by a known distance by which the controller can compute the composition of the expired gases.

In some embodiments, an anesthesia machine of the present teachings can comprise inlet and outlet retractable hose barb ports. In various configurations, these barb ports can be compatable with numerous oxygen tubing inside diameters that are known in the art.

In some embodiments, an anesthesia machine of the present teachings can comprise in the liquid inhalational anesthetic reservoir laser etched microgrooves in a radial pattern. In various configurations, these grooves can facilitate liquid movement through capillary action towards the exit hole, and can thereby render the device capable of being used in an inverted position.

The structure of an anesthesia machine can be described as follows in the following non-limiting exemplary figures.

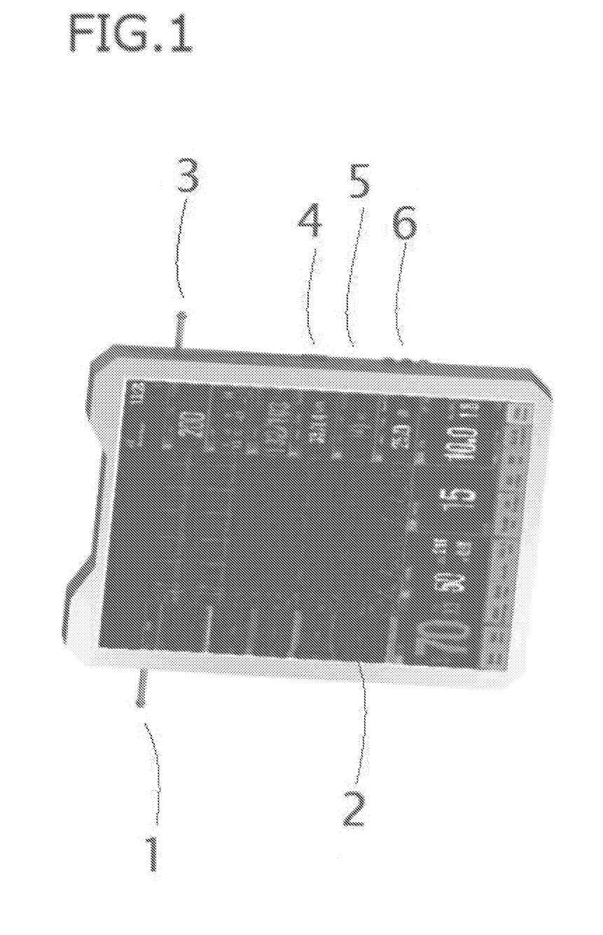

With reference to FIG. 1, 1 and 3 are gas inlet and outlet ports, respectively, each comprising a retractable ¼ inch hose barb. Each hose barb is capable of receiving standard oxygen tubing. 1 can be connected by hosing to a gas source such as an oxygen tank; 3 can be connected by hosing to a patient. 2 is a 180 mm×130 mm capacitive touch screen GUI. 4 is a micro-B USB port for external communication and power charging. 5 is an Sa02 port for infra-red transillumination of patient finger for oxygen saturation analysis. 6 is a 3-axis electrocardiography ports that can be color coded.

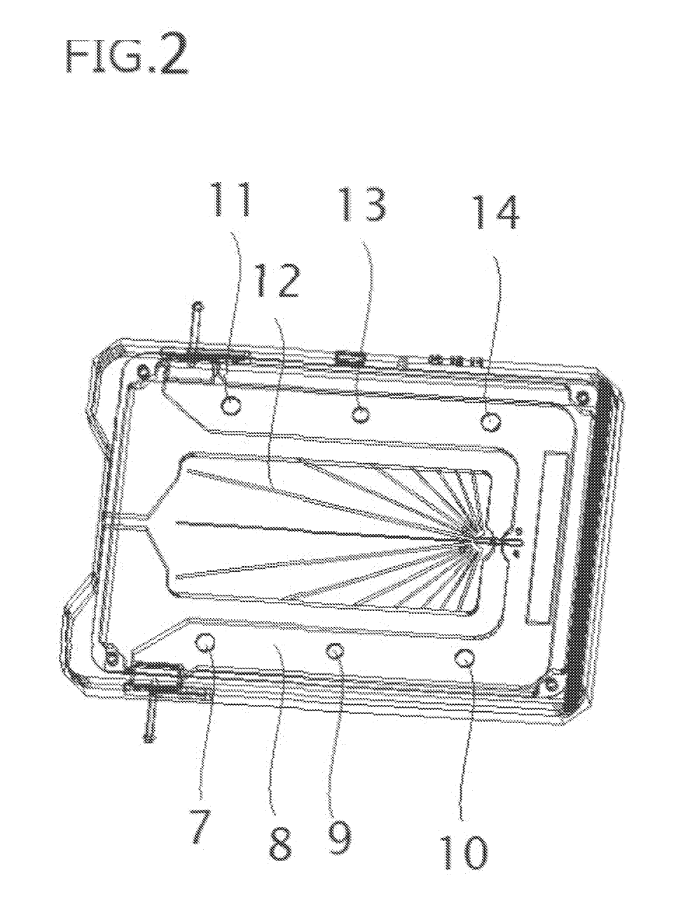

With reference to FIG. 2, 7 is the location of a thru hole for acoustic sensor L1 to detect presence of moving gases by speed and time-of-flight with acoustic sensor L2 (10). 8 is the main corridor through which the carrier gas enters and mixes with the evaporated inhalational anesthetic. 9 is the location of the thru hole for temperature sensor (thermistor) T1 sealed in place to allow direct contact with carrier gas. 10 is the location of sensor L2 which works with L1 to determine composition of and relative speed of the incoming carrier gas. 11 is the location of acoustic sensor L3 which allows a cross check with L4 (14) to confirm evaporation of agent and composite percent by volume with the carrier gas prior to exit to the patient. 12 is a laser etched micro groove that employs capillary action to migrate liquid inhalational anesthetic to the exit hole regardless of the unit's orientation. 13 is the location of the second temperature device (thermistor) T2 that measures the change in carrier gas temperature indicating successful evaporation of agent, or triggers alarms if none is detected. 14 is position of acoustic sensor L4 which works in tandem with L3 (11) to confirm agent evaporation and to adjudge composition of the diluent gases.

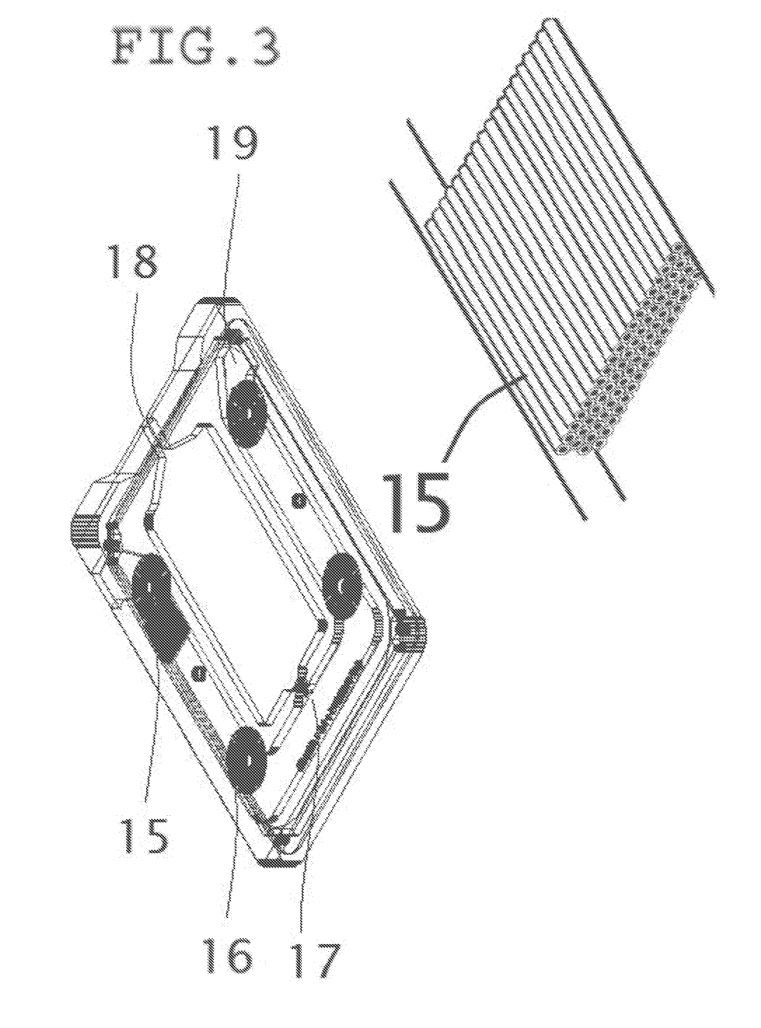

With reference to FIG. 3, 15 shows the stacked micro tubes that induce a laminar flow of the incoming carrier gas for fine control of evaporation. A magnified view of the stacked micro tubes (15) is shown in the inset. 16 illustrates a 2-dimensional printed graphene inductor coil that responds to a cylindrical magnet positioned in the center on a thin film of polyvinylchloride which covers the thru hole to the carrier gas corridor. L1 thru L6 use this as acoustic sensors. 17 shows the location of the liquid transfer solenoid which reaches into the liquid reservoir and accepts a specific amount of agent then communicates it to the carrier gas flow stream on each stroke. In some embodiments the volume of this transfer can be approx. 1.74 cubic millimeters (1.74 microliters) which translates into 292 cubic millimeters (292 microliters) of evaporated gas per stroke. 18 shows a liquid port to the reservoir, which can be filled with a standard 10 ml syringe. After filling the reservoir the operator would unscrew the syringe leaving the needle in place (pierced through the rubber stopper allowing room air to enter the reservoir as the liquid is carried out at the bottom preventing a vacuum from occurring. 19 shows the exit port to patient.

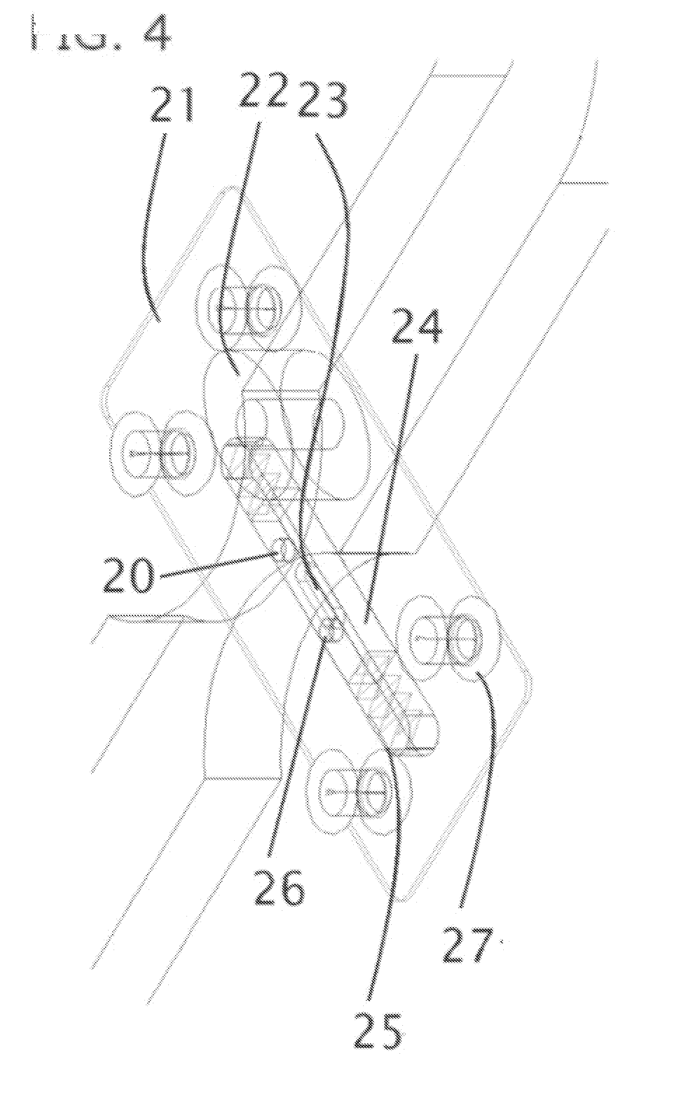

With reference to FIG. 4, 20 is the 1 mm entry portal through which liquid inhalational anesthetic passes into trough 23 for transfer to the lower carrier gas corridor. 21 is the cover plate that seals in the sliding actuator bar which is free to slide up and back during action. 22 is a standard wire wound inductor coil rated to impart sufficient electromotive force to attract the solenoid bar which is chromed iron that then opposes the two corrogated springs that hold the bar in the normally off position. 23 shows the trough milled into the solenoid bar that shuttles the liquid drop (1.74 mm³) during operation. When activated, the trough is carried up to expose itself to the standing liquid and fills with the agent. When relaxed, it carries this discrete quantum of measured liquid to the exit hole into the lower corridor. The hole positions prevent formation of opening from the reservoir to the corridor under any circumstance. When one portal is aligned, the other is obstructed. 24 is the chromed iron bar polished and milled to slide freely in the slot with the two springs opposing it. 25 illustrates the corrogated stainless steel spring which provides positioning and maintains the positioning after thousands of cycles of use. 26 is the exit portal also 1 mm where the inhalational anesthetic enters the corridor. 27 shows the rivets used to attach the cover plate ensuring a sealed actuation area.

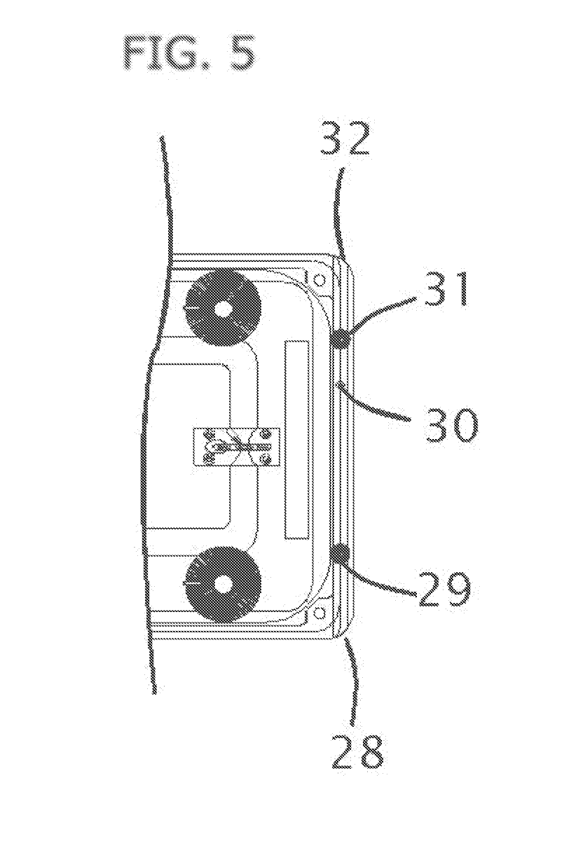

With reference to FIG. 5, 28 is the exit port for returned composite gases from a pop-off assembly attached to a patient breathing system. From here the gases travel to the waste scavenger system. 29 is the location of a smaller acoustic sensor L5 which operates in tandem with acoustic sensor L6 (31) to evaluate the exhaled gases from the patient to determine the C02 (end tidal) as well as the data for plotted wave forms to the GUI. 30 is the T3 temperature sensor used in the analysis of the feedback gases. 31 represents L6 acoustic sensor. 32 is the inlet port for the feedback gases, receives a known in the art sampling cannula attached to the pop off valve.

The following non-limiting example sets out an exemplary use of a device of the present teachings.

1) On power up with fully charged 500 mAh lithium battery, system master control unit performs the following diagnostics for operation.
2) GUI main page displayed.
3) Check network signal available. Read resistive value liquid reservoir.
4) Read values of L1-L4 to determine flowing carrier gas.
5) When L1 reaches threshold, GUI displays carrier presence.
6) L2 transmits “Train of 4” signals at 50 Khz.
7) L1 reads the delay of the “Train of 4” sawtooth wave forms and computes travel time.
8) L1 magnitude is computed as velocity.
9) T1 reading provides carrier gas temperature.
10) GUI displays carrier composition, speed and temperature.
11) System waits user input for desired delivery percentage.
12) User input is variable X
13) Var X determines the frequency of actuation of the agent sliding solenoid.
14) L3 transmits “Train of 4” and is read by L4 to determine presence of evaporated agent.
15) T2 corroborates this presence with a lower reading than T1.
16) Measured output sent to GUI and reconfirmed every 5 seconds during operation.
17) Changes in user setting for output affect var X interrupt.
18) If magnitude of L1 falls below threshold, solenoid actuation stops and alarm condition and messaging sent to GUI.
Interrupt request list:

Out of threshold L1 (carrier gas speed)

Out of threshold t1 (carrier gas too cold/hot)

Absence of liquid agent

Insufficient battery/wall supply voltage

User request higher than allowed for agent (MAC+short duration above)

Network interface interruption (for nonqualified sole user)

If on startup, T1 is too low, a warm up period is required to generate sufficient heat for operation.

19) A disposable common oxygen tube attaches to the bottom ports of the machine that allows exhaled gases from the patient via the pop-off valve to flow through the lower corridor where inductor coils L5 and L6 can transmit and receive a 150 KHz train of 4 signals for composition analysis to include phase shift, temporal delay and temperature T3.
20) Data acquired through the lower corridor is up loaded to the main server for collective quantitative analysis and a reasonable approximation of the combined respiratory gases can be sent back to the hand held unit giving the user a view of the patient's disposition.

All references cited are incorporated by reference.

What is claimed is:

1. An anesthesia gas delivery device, comprising:
a cover plate;
a gas inlet for a carrier gas;
a gas outlet for a diluent anesthetic gas;
a gas corridor in fluid communication with and extending between the gas inlet and the gas outlet, said corridor enveloped by walls;
a first acoustic sensor situated in the gas corridor adjacent to the gas inlet;
a second acoustic sensor situated in the gas corridor downstream of the first acoustic sensor;
a third acoustic sensor situated in the gas corridor downstream from the second acoustic sensor;
a fourth acoustic sensor situated in the gas corridor downstream from the third acoustic sensor and adjacent to the gas outlet;
a reservoir comprising a reservoir housing for a liquid inhalational anesthetic;
means for transferring a sample of a liquid inhalational anesthetic from the reservoir to the gas corridor between the second acoustic sensor and the third acoustic sensor; and
a controller
wherein the first acoustic sensor produces a first sensor signal indicative of velocity and composition of gas introduced to the gas corridor at the gas inlet, the second sensor produces a second sensor signal indicative of the composition and velocity of gas upstream from the means for introducing an inhalational anesthetic, the third sensor produces a third sensor signal indicative of composition of gas downstream from the means for introducing an inhalational anesthetic, the fourth acoustic sensor produces a fourth signal indicative of composition of gas at the gas outlet, and the controller receives the first, second, third and fourth sensor signals and computes a composition of diluent anesthetic gas.

2. The anesthesia gas delivery device of claim 1, wherein the cover plate comprises aluminum.

3. The anesthesia gas delivery device of claim 1, wherein the acoustic sensors are ultrasound sensors.

4. The anesthesia gas delivery device of claim 1, further comprising a first thermistor situated between the first acoustic sensor and the means for introducing the liquid inhalational anesthetic, and a second thermistor situated between the third acoustic sensor and the gas outlet.

5. The anesthesia gas delivery device of claim 1, further comprising a means for vaporizing the sample of the liquid inhalational anesthetic.

6. The anesthesia gas delivery device of claim 1, wherein the means for transferring a sample of a liquid inhalational anesthetic from the reservoir to the gas corridor comprises:
a ferromagnetic bar positioned between the corridor and the reservoir, wherein the bar comprises a slot enclosing a volume of about 1 microliter, from 1 to 30 microliters, or about 30 microliters;
springs supporting the ferromagnetic bar;
a solenoid coil that moves the bar upon energizing;
a first portal in the reservoir housing that is in fluid communication with the slot when the ferrite bar is energized; and
a second portal in the corridor wall that is in fluid communication with the slot when the ferrite bar is not energized.

7. The anesthesia gas delivery device of claim 1, further comprising a heat patch whereby liquid inhalational anesthetic in the corridor is vaporized.

8. The anesthesia gas delivery device of claim 1, further comprising a second corridor, a second inlet, a second outlet a fifth acoustic sensor and a sixth acoustic sensor, whereby composition of exhalation gas from a subject is analyzed.

9. The anesthesia gas delivery device of claim 1, wherein the reservoir comprises one or more grooves.

10. The anesthesia gas delivery device of claim 9, wherein the one or more grooves are etched grooves.

11. The anesthesia gas delivery device of claim 9, wherein the one or more grooves extend radially from the means for introducing a sample.

12. The anesthesia gas delivery device of claim 9, wherein the reservoir further comprises a resistive wire.

13. The anesthesia gas delivery device of claim 1, further comprising a capacitive touch screen.

14. The anesthesia gas delivery device of claim 1, further comprising a USB port.

15. The anesthesia gas delivery device of claim 1, further comprising a spO₂ sensor port.

16. The anesthesia gas delivery device of claim 1, further comprising axial ports for electrocardiography.

17. The anesthesia gas delivery device of claim 1, further comprising internet connectivity.

18. The anesthesia gas delivery device of claim 1, wherein the gas inlet and the gas outlet each comprises a barbed hose connector.

19. The anesthesia gas delivery device of claim 18, wherein each barbed hose connector is a retractable barbed hose connector.

20. A device for dispensing a pre-determined volume of a liquid from a reservoir, comprising:
a reservoir comprising walls and enclosing a liquid;
a destination location
a ferromagnetic bar positioned between the corridor and the reservoir, wherein the bar comprises slot enclosing a volume of about 1 microliter, from 1 to 10 microliters, or about 10 microliters;
springs supporting the ferromagnetic bar;
a solenoid coil that moves the bar upon energizing;
a first portal in the reservoir housing; and
a second portal in the corridor wall.

21. A device in accordance with claim 20, wherein the ferromagnetic bar comprises a ferromagnetic material selected from the group consisting of ferrite, stainless steel and chromed iron.

22. A method of anesthetizing a subject, comprising:
mixing a carrier gas with an inhalational anesthetic using the device of claim 1 thereby forming a diluent gas; and
supplying the diluent gas to the subject.

23. A method of anesthetizing a subject in accordance with claim 22, wherein the carrier gas is selected from the group consisting of oxygen, nitrous oxide, air, helium and a combination thereof.

24. A method of anesthetizing a subject in accordance with claim 22, wherein the inhalational anesthetic is selected from the group consisting of isoflurane, halothane, sevoflurane, ethrane, desflurane and a combination thereof.

25. A method of anesthetizing a subject, comprising:
mixing a carrier gas with an inhalational anesthetic using the device of claim 6 thereby forming a diluent gas; and
supplying the diluent gas to the subject.

26. A method of anesthetizing a subject in accordance with claim 25, further comprising evaluating composition of exhalation gas.

27. A method of anesthetizing a subject in accordance with claim 25, wherein the evaluating composition of exhalation gas comprises evaluating carbon dioxide content of the exhalation gas.
